(19) United States
(12) Patent Application Publication
(10) Pub. No.: US 2019/0380625 A1
(43) Pub. Date: Dec. 19, 2019

(54) METHOD FOR EVALUATING MANUAL DEXTERITY

(71) Applicants: Universite Paris Descartes, Paris (FR); Sensix, Poitiers (FR)

(72) Inventors: Pavel Lindberg, Paris (FR); Maxime Teremetz, Paris (FR); Marc Maier, Paris (FR)

(73) Assignees: Universite Paris Descartes, Paris (FR); Sensix, Poitiers (FR)

(21) Appl. No.: 15/575,146

(22) PCT Filed: May 19, 2016
(86) PCT No.: PCT/EP2016/061191

§ 371 Date: Nov. 17, 2017

(30) Foreign Application Priority Data
May 19, 2015 (EP) 15305752.6

(51) Int. Cl. A61B 5/11; A61B 5/22; A61B 5/00

(52) U.S. Cl. CPC A61B 5/1125; A61B 5/1126; G01L 1/04; A61B 5/7475; A61B 2505/09; A61B 5/225

(57) ABSTRACT

The present invention relates to a new method for quantifying key components of manual dexterity. The present invention also provides methods for diagnosing impaired upper limb and/or hand function in patients based on how these components are affected.

### INTRODUCTION

A high degree of manual dexterity is a central feature of the human upper limb. A rich interplay of sensory and motor components in the hand and fingers allows for independent control of fingers in terms of timing, kinematics and force.

Manual dexterity allows fine control in grasping and manipulating small objects. Across species, manual dexterity is most evolved in humans [1]. This high degree of manual dexterity is made possible by specializations in hand morphology (skeletal, muscular) and neural control (corticospinal tract) [2]. Together these specializations allow for purposeful goal- and object-oriented manual control. There is, however, no consensus on how dexterity should be operationally defined and quantified. Although historically an ‘index of dexterity’ was developed (mainly for phylogenetic considerations [3]), it has become clear that behavioral dexterity cannot be defined by a single variable. Consequently, several studies have outlined key components of manual dexterity in terms of motor control: (i) Control of force, such as the capacity to control the force in each finger [4], in precision grip [5], in power grip [6,7] and during grasp-and-lift tasks [8], (ii) Finger independence, i.e. the capacity to move the fingers independently of each other [9,10]. (iii) Timing aspects, illustrated by the capacity to synchronize finger movements [11] and (iv) Motor sequence performance, i.e., activation of different fingers in a temporal sequence [12,13]. However, a simultaneous description of such components is lacking in patients with neurological upper limb impairments.

Stroke is the first cause of acquired handicap in adults and about 50% of stroke survivors have impaired upper limb and hand function in the chronic phase [14,15], which impacts strongly on activities of daily living and on independence. Most of the above outlined dexterity components have been studied in stroke patients: (i) In terms of force control: post-stroke upper limb weakness is prevalent [14,16,17] and a decrease of accuracy in force control has also been reported (power grip [18]; precision grip: [19]; grasp-and-lift tasks: [20,21]). (ii) Studies have also shown decreased independence of finger movements and increased motor overflow after stroke [22,23]. (iii) Timing is also impaired after stroke: repetitive finger movements are slowed down and regularity is decreased [24-26]. (iv) Execution of sequential finger movements can also be compromised in stroke [27]. Therefore, manual dexterity can be impaired by decreased control of force, independence of finger movements, timing or finger sequencing.

In spite of evidence of impaired components of dexterity, clinical practice in terms of diagnosis and treatment of manual dexterity relies essentially on ‘functional’ measures and scales. Although largely applied, some of these scales are subjective, show questionable validity and reliability [28,29] and some have high measurement error [30]. This may hamper detection and evaluation of motor deficits and affect evaluation of spontaneous or treatment-specific recovery [31,32]. Most critically: usually these methods have investigated only one of the components of dexterity. It remains therefore unclear to what degree each of these components is affected in hemiparetic patients with impaired hand function.

Thus there is still a need for a reliable and sensitive method for detecting upper limb and/or hand function impairment.

### DESCRIPTION

The inventors have now developed a new method for quantifying key components of manual dexterity and to describe how these components are affected in patients with impaired upper limb and/or hand function.

By “manual dexterity”, it is herein referred to the ability to perform accurate and coordinated hand and finger movements. More specifically, manual dexterity as used herein comprises the following motor control components: (i) control of force, (ii) finger independence, (iii) timing aspects, and (iv) motor sequence performance. By “control of force”, it is herein referred to the capacity to control the force in each finger, in precision grip, in power grip and during grasp-and-lift tasks. The term “finger independence”, as used herein, refers to the capacity to move the fingers independently of each other. As used herein, the term “timing aspects” refers to the capacity to synchronize finger movements. Finally, “motor sequence performance”, as used herein, refers to the activation of different fingers in a temporal sequence.

In a first aspect, the invention provides a method for evaluating manual dexterity in a subject, said method comprising assessing the performance of the subject in the three following tasks:
(i) The Finger Force-Tracking;
(ii) The Single finger tapping; and
(iii) The Multi-finger tapping.

In a preferred embodiment, the method comprises assessing the performance of a subject in a fourth task, The Sequential finger tapping, in addition to the previous three tasks.

The method of the invention is particularly advantageous. First, it must be emphasized that the method of the invention is representative of the clinical situation, since a good correlation has been observed between the responses to the tasks of the invention and the scores in recognized clinical tests such as the Action Research Arm Test (ARAT).

In addition, the method of the invention is both more sensitive and more discriminatory than the methods of the prior art. Whereas the methods of the prior art only tested individual components of manual dexterity, the method of the invention relies on the determination of an ensemble of four parameters, which assess each of the components of manual dexterity. The method of the invention thus provides a more thorough and complete image of the manual capacities of a subject than the methods of the prior art. This is particularly advantageous since patients vary in their impairments. Indeed, the present inventors have shown that patients are not equally affected across different components of the manual dexterity. Some patients may fail to display a deficiency in one or more of the components of manual dexterity, while still clearly showing a weakening in the other components. Such patients would thus not be recognized as deficient in hand function by some of the tests of the prior art and, as a consequence, would not be adequately treated. On the other hand, such patients are readily identified by the method of the invention and can thus be treated, i.e., they can undergo rehabilitation.

Moreover, the method of the invention assesses the force exerted by each of the fingers. Methods of the prior art had never sought to evaluate how finger force was affected in patients with impaired upper limb and/or hand function. By comparison, all of the parameters measured by the method of the invention have a force component, which means that defects associated with a deficiency in exerting or controlling finger force can be readily detected.

The present inventors have developed three separate tasks (i-iii), with an optional fourth (iv), in order to assess the different components of manual dexterity:
(i) The Finger Force-Tracking;
(ii) The Single finger tapping;
(iii) The Multi-finger tapping; and
(iv) The Sequential finger tapping.

The Finger Force-Tracking task consists of a visuo-motor task of finger force control. The subject is instructed to apply defined forces on a piston with the finger, and the force actually exerted is recorded. The Finger Force-Tracking task comprises the steps of:
a) providing instructions to the subject to exert a defined force (the target force) by a unique finger on a piston; and
b) measuring the force exerted on the piston.

The finger used in the Finger Force-Tracking can be any one of the four fingers. Preferably, said finger is the index or the middle finger. More preferably, said finger is the index.

In this task, the subject will be expected to vary the force exerted in response to instructions. The inventors have shown that patients with impaired upper limb and/or hand function display decreased force control in this test compared to healthy subjects. In particular, said patients are significantly affected in the response to said signal in comparison to healthy subjects.

Preferably, the instruction of step a) is a visual cue. Said visual cue can be any type of visual stimulus which signals the subject to modify the force exerted on the piston. The modification of the force may be an increase or a decrease. Accordingly, the visual cue must be capable of signaling that said force must be increased or decreased. In a preferred embodiment, the visual cue may signal successive modifications of the force exerted on the piston. For example, a signal to increase said force may be followed by a signal to maintain said force constant or a signal to decrease said force. Likewise, a signal to decrease said force may be followed by a signal to maintain said force constant or a signal to increase said force. Also, a signal to maintain said force constant may be followed by a signal to increase said force or a signal to decrease said force. The skilled person will thus easily realize that it is possible to construct defined sequences of signals and to measure the force actually exerted by the subject in response to these signals throughout the sequence. A variation in the nature (increased, decreased or constant force) of the modifications signaled in these sequences increases the specificity of the response to the task since a defect in force control is expected to show up in response to different signals. The inventors have indeed shown that patients with impaired upper limb and/or hand function are significantly affected during both the “increased” and “constant” phases. Likewise, the repetition of the measurements during the sequence greatly increases the specificity of the task.

The visual cue is preferably a signal appearing on a screen. For example, said visual cue is a line, wherein said line is horizontal when the force must be constant, ascending when the force must be increased, and descending when force must be decreased. More preferably, the visual cue of the invention may include a cursor, whose position on the screen is controlled by the force exerted by the subject on the piston with the finger. In this embodiment, the subject has to follow the line with the cursor by exerting a force with the finger until a target force, represented by the line, is reached.

This task thus assesses the capacity of the subject to control the force exerted by the finger. A subject who is deficient in this regard will apply a force which is different from the target force. It may be for example higher or lower than said target force.

Preferably, a tracking error may be calculated as the root-mean-square error between the actual applied force and the target force. According to a preferred embodiment, the Finger Force-Tracking comprises a further step of calculating the tracking error as the root-mean-square error between the actual applied force and the target force. Not only can the intensity of the force applied be measured, but also the temporal response to stimuli, or the duration of said response. According to a preferred embodiment, the Finger Force-Tracking comprises a further step of measuring the time between the visual cue and the actual exertion of said force on the piston. This embodiment thus encompasses all situations wherein the force applied varies as the result of an instruction received by the subject. Specifically, this embodiment encompasses the situation wherein the subject is instructed to stop applying any force, i.e., to apply a force of 0 N: in this situation, the time elapsed between the signal and the release of the force to return to a resting force of 0 N is measured. According to another preferred embodiment, the Finger Force-Tracking comprises a further step of measuring the time during which the force is exerted. Both of them are affected in patients with impaired upper limb and/or hand function. According to a further preferred embodiment, the Finger Force-Tracking comprises a further step of measuring the time between the visual cue and the actual exertion of said force on the piston and a further step of measuring the time during which the force is exerted. In yet a further preferred embodiment, the Finger Force-Tracking comprises instructing the subject to release the applied force to return to a resting force of 0 N; according to this embodiment, a release duration is computed as the time taken to reduce the force from 75% to 25% of the target force.

The Single finger tapping task consists of repetitive tapping with one finger. This task aims at testing the control of timing of the subject. The subject is instructed to tap a piston with a specific finger at a defined rate and the taps on the pistons are detected. Thus, the task comprises the steps of:

a) providing instructions to the subject to tap a piston with a specific finger at a defined rate; and
b) detecting the taps on the piston.

Because some impairment may be more apparent when the subject is instructed to tap at a lower or a higher rate, it may be advantageous to vary the rate of tapping. Thus, in a preferred embodiment, the task may include a further step of repeating steps a) and b) at a different rate.

The finger in the method of the invention can be any of the four fingers of the hand (index, middle finger, ring finger and little finger). In order to detect other impairments, or to accumulate more data and add both specificity and sensitivity, it may be advantageous to repeat steps a) and b) with another finger. According to this preferred embodiment, the task may include a further step of repeating steps a) and b) with a different finger. Even more preferably, the task may include further steps of c) repeating steps a) and b) at a different rate, and d) repeating steps a) to c) with a different finger.

A tap is a discrete event, corresponding to one pressing of a piston by a finger. Detecting the tap on the piston in this task involves determining the force exerted by the subject on the piston. In order to avoid that spurious interactions between fingers and pistons are recorded as intentional taps, it is advantageous that a minimal force be exerted by the subject on the piston for a tap to be recorded. In addition, this allows finger force control to be tested.

Since each tap is detected, it is possible to measure the rate of tapping. Preferably, the rate of tapping is compared with the rate of instructions. In this task, as in the previous and the following tasks, each instruction is expected to be followed by a tap on the piston by the subject. Hence the frequency of the instructions determines the rate of tapping. In other words, if the task is completed successfully, there should be no tap without a prior instruction and each instruction should be followed by a tap. In this case, the frequency of tapping should match closely the frequency of instructions. This is indeed what was observed by the inventors when the task was performed by healthy subjects. On the other hand, patients impaired in upper limb and/or hand function show reduced ability to follow the imposed tapping rate. In a preferred embodiment, the task comprises a further step of measuring the rate of tapping. In a further preferred embodiment, the task comprises a further step of comparing the rate of tapping and the rate of instructions.

Other parameters can also be measured which are informative. For example, taps by a finger other than the specified finger in the absence of a concomitant tap by said specified finger (unwanted taps) indicate lack of motor control, whereas taps by a finger other than the specified finger concomitant with a tap by said specified finger (overflow taps) is indicative of motor overflow to fingers not involved in the task. Both unwanted finger taps and overflow taps in patients impaired in upper limb and/or hand function such as e.g., patients recovering from a stroke. In a preferred embodiment, the step of detecting the taps on the piston further includes detecting taps by a finger other than the specific finger of step a) in the absence of a concomitant tap by said specific finger. In another preferred embodiment, the step of detecting the taps on the piston further includes detecting taps by a finger other than the specific finger of step a) concomitant with a tap by said specific finger. In yet another preferred embodiment, the step of detecting the taps on the piston further includes detecting taps by a finger other than the specific finger of step a) in the absence of a concomitant tap by said specific finger and detecting the taps on the piston further includes detecting taps by a finger other than the specific finger of step a) concomitant with a tap by said specific finger.

In a further preferred embodiment, the task comprises a further step of computing detecting taps by a finger other than the specific finger of step a) in the absence of a concomitant tap by said specific finger. In another further preferred embodiment, the task comprises a further step of computing taps by a finger other than the specific finger of step a) concomitant with a tap by said specific finger. In yet another further preferred embodiment, the task comprises a further step of computing taps by a finger other than the specific finger of step a) in the absence of a concomitant tap by said specific finger and computing taps by a finger other than the specific finger of step a) concomitant with a tap by said specific finger.

Preferably, the instructions received by the subject are visual or aural cues. Said visual or aural cues are any type of visual or aural stimuli which signal the subject to tap the piston. It may be advantageous to make the subject repeat the tapping at a defined rate but without any instruction, either aural or visual. In a preferred embodiment, the task comprises a further step of making the subject repeat step a) without any instruction.

The Multi-finger tapping task consists of simultaneous tapping with different finger configurations in response to instructions. The subject is instructed to tap a piston with one or more specific fingers simultaneously and the taps on the pistons are detected. Thus, the task comprises the steps of:

a) providing instructions to the subject to tap a piston with one or more fingers simultaneously; and
b) detecting the taps on the piston.

In a preferred embodiment, the Multi-finger tapping task consists of simultaneous tapping with different finger configurations in response to instructions, wherein one finger is individually tapping on one piston as shown on FIG. 1. The subject is instructed to tap one or more pistons with one or more specific fingers simultaneously and the taps on the pistons are detected. Thus, the task comprises the steps of:

a) providing instructions to the subject to tap one or more pistons with one or more fingers simultaneously; and
b) detecting the taps on the piston.

The finger in the method of the invention can be any of the four fingers of the hand (index, middle finger, ring finger and little finger). The subject may be instructed to tap with any combination of between one and four fingers. Preferably, the method of the invention involves tapping with either a single finger or a combination of two fingers: index/middle finger, index/ring finger, index/little finger, middle finger/ring finger, middle finger/little finger, and ring finger/little finger. More preferably, the method is repeated with every combination of one and two fingers.

A tap is a discrete event, corresponding to one pressing of a piston by a finger. Detecting the tap on the piston in this task involves determining the force exerted by the subject on the piston. In order to avoid that spurious interactions between fingers and pistons are recorded as intentional taps, it is advantageous that a minimal force be exerted by the subject on the piston for a tap to be recorded. In addition, this allows finger force control to be tested.

In an embodiment, the task comprises a further step of repeating steps a) and b) n times, n being an integer between 0 and 64. Such repetition makes it possible to compare the sequences of taps with the sequences of instructions. Preferably, said comparison comprises computing correct and incorrect taps. Even more preferably, said comparison comprises computing correct and incorrect tap sequences. A “correct tap sequence” is a sequence in which all the taps are correct. An “incorrect tap sequence” is a sequence containing at least one incorrect tap. A “correct” tap is a tap made by the finger as instructed. Any other tap, i.e., a tap different from the required target tap, is an “incorrect” tap. Such errors can further be categorized as missing taps, i.e., omissions (no tap in response to instruction), or as extra-finger taps, i.e., additional taps with other finger(s) than the one(s) of the task. Omissions and extra-finger taps are computed. These parameters are significantly greater in patients impaired in upper limb and/or hand function than in healthy subjects. In a preferred embodiment, the task comprises a further step of detecting absent taps in response to instructions, and/or additional taps with other finger(s) than the one(s) of the task. In a further preferred embodiment, the task comprises a further step of computing absent taps in response to instructions, and/or additional taps with other finger(s) than the one(s) of the task.

The Sequential finger tapping task is a multiple-tap finger sequence involving the four fingers. In this task, the subject is instructed to tap pistons with the four fingers in a defined sequence. Taps are detected and the sequence of taps is compared to the sequence of instructions. Hence, the subject is provided with a defined sequence of instructions to tap pistons with the four fingers, wherein each instruction of the said sequence is expected to be followed by a tap on a piston. A tap is a discrete event, corresponding to one pressing of a piston by a finger. Detecting the tap on the piston in this task involves determining the force exerted by the subject on the piston. In order to avoid that spurious interactions between fingers and pistons are recorded as intentional taps, it is advantageous that a minimal force be exerted by the subject on the piston for a tap to be recorded. In addition, this allows finger force control to be tested.

In this task, as in the two following tasks, each instruction is expected to be followed by a tap on the piston by the subject. In other words, if the task is completed successfully, there should be no tap without a prior instruction and each instruction should be followed by a tap. Preferably, the instructions received by the subject are visual cues. Said visual cues are any type of visual stimuli which signal the subject to tap the corresponding piston. Since the instructions are provide in a sequence, said visual cues thus appear in a sequence, following a predefined order. If the task is successfully completed, each of the instructions should be followed by a tap on the correct piston and the sequence of tapping should be identical to the sequence of instructions. Indeed, this is how healthy subject perform. However, the inventors have observed that patients with impaired upper limb and/or hand function are affected in their capacity in effecting this task.

Preferably, the steps of providing instructions to the subject to tap according to a sequence and of detecting said taps are repeated. It has been previously shown that motor learning capacities are intact but slowed in patients recovering from a stroke. The repetition of said steps allows detecting such slowed motor learning capacities. According to this embodiment, the Sequential finger tapping task comprises steps of:

a) instructing the subject to tap pistons with the four fingers in a defined sequence;
b) detecting the taps on the pistons;
c) repeating steps a) and b) n times, wherein n is an integer comprised between 0 and 15; and
d) comparing the sequence of taps to the sequence of instructions.

In a preferred embodiment, the Sequential finger tapping task comprises the further steps of repeating the sequence of step a) without instructions and detecting the taps. This specific embodiment aims at testing the ability to recall the sequence learned during the previous learning phase. To perform this additional step, the subject is commanded to repeat the tapping sequence of step a) without being provided any instruction before each tap of said sequence.

According to this embodiment, the Sequential finger tapping task comprises the steps of:

a) providing instructions to the subject to tap pistons with the four fingers in a defined sequence;
b) detecting the taps on the pistons;
c) repeating steps a) and b) n times, wherein n is an integer comprised between 0 and 15;
d) commanding the subject to tap the sequence of step a) without instructions;
e) detecting the taps on the pistons; and
f) comparing the sequence of taps to the sequence of instructions.

According to a particular embodiment, the Sequential finger tapping task comprises the steps of:

a) providing instructions to the subject to tap piston with the four fingers in a defined sequence;
b) detecting the taps on the pistons;
c) repeating steps a) and b) n times, wherein n is an integer comprised between 0 and 15;
d) commanding the subject to tap said sequence of step a) without being provided any instruction before each tap of the said sequence;
e) detecting the taps on the pistons; and
f) comparing the sequence of taps to the sequence of instructions.

Preferably, comparing the sequences comprises computing correct and incorrect tap sequences. A “correct tap sequence” is a sequence in which all the taps are correct. An “incorrect tap sequence” is a sequence containing at least one incorrect tap. A “correct” tap is a tap made by the finger as instructed. Any other tap, i.e., a tap made by another finger, an extra tap (a tap without any preliminary instruction), or an absent tap, is an “incorrect” tap. For example, if the subject is instructed to tap with the middle finger but taps with the index, it is recorded as an incorrect tap. Likewise, when the subject taps with the middle finger and then adds an extra, unsolicited tap: the second tap is an incorrect tap. Also, if the subject is instructed to tap twice with the middle finger, but only taps once, the absent tap is an incorrect tap. More preferably, the number of consecutive correct taps within parts of an incorrect sequence is computed.

The method of the invention thus comprises assessing the performance of the subject in at least three of the four tasks described above. Preferably, said tasks are: Force tracking, Single finger tapping, and Multi-finger tapping. More preferably, all four tasks are assessed in the method of the invention.

It can be practiced with any device suitable for performing the above tasks.

Preferably, the method of the invention is practiced with a device (the Finger Force Manipulandum, FFM) which was designed by the inventors to quantitatively assess several key components of dexterity. The FFM is described in EP 2 659 835 B1 and is commercially available (Sensix, France). It is both a force-torque sensor and a displacement sensor. The FFM provides a device for ease of use, high adaptability, for measurements in both isometric regime that non isometric regime. It allows the force-displacement study of finger motion one after the other or altogether. This diagnosis tool integrates a biofeedback in real time which provides accurate diagnosis of fingers force and flexion.

The FFM is a device for quantifying the independence of the fingers, which incorporates a set of pistons inserted in an accommodating structure and allowing the said pistons to be pressed by the fingers of a hand, except using the thumb, subject to a movement against a return system that brings it into contact with force sensors transmitting information items to an information-recording device. The FFM is designed to be handled by a user patient and held in the palm of the hand. The device comprises a reception structure for subassemblies, wherein each subassembly is positioned vertically and includes a sleeve in fixed position that is internally hollow for the passage of a piston. Said piston is mounted so as to slide freely within the sleeve. The piston is hollow and threaded internally to accommodate a screw. This screw allows adjusting the piston in relation to a sensor component which is positioned underneath, against a return and compression system.

The FFM is a simplified device for measuring and quantifying the independence of the fingers both at the level of motion (kinematics) and at the level of the applied force (dynamic). It is easily usable by a subject, even if said subject suffers from hemiparesis. Indeed, the inventors observed that stroke patients were able to use the FFM and perform most of the tasks of the method of the invention with the FFM.

Indeed, the inventors have shown the FFM allows for extraction and quantification of key control variables of manual dexterity in a subject. The FFM enables measuring the independence of the fingers and that is easily manageable by the operator or user, and can be held in the palm of the hand and manipulated by the fingers of the user. The FFM also allows the analysis of forces in different tasks, but not limited in relation to the contraction of a finger isolation that leads to an involuntary contraction of neighboring fingers. As described in EP 2 659 835, the FFM allows both isometric and non isometric measurements, which leads to a much more complete and independent measure of the fingers of the subject. The FFM thus allows measuring easily and precisely the performance of the patient in all the four tasks of the method of the invention.

In daily clinical practice, upper extremity impairments, i.e., injuries to the hand, wrist, elbow and shoulder, are frequently encountered. Upper extremity disorders fall into three general categories: inflammatory disorders, compressive neuropathies and nonspecific complaints. With the exception of the inflammatory disorders (e.g., epicondylitis, tenosynovitis, tendinitis and arthritis), there are currently no definitive diagnostic criteria for upper extremity impairment. The present invention provides a way to establish such a diagnostic.

In another aspect, the invention thus relates to a method of diagnosing upper limb and/or hand impairment in a subject. According to the method of the invention, the performance of the subject in each of the tasks is compared to a reference performance. Preferably, said reference performance is the performance of a healthy subject.

As explained above, the inventors have found that there are variations in individual dexterity profiles. Some patients, who are clinically identified as affected in hand function, can perform some of the tasks as well as healthy subjects, but display impaired performance in the others. Therefore, a subject is diagnosed as being healthy only if the performances of the said subject are at least equal to the performances of the reference healthy subject in each and every of the tasks tested, i.e., at least three, preferably four. If said subject displays performances in at least one of the tasks below the reference performance, said subject is diagnosed as suffering from upper limb and/or hand impairment.

A “subject” as used herein refers to a human being. Said subject may be affected by another condition, said other condition being known for affecting the function of the upper limb and/or hand. Such conditions include stroke and cerebral palsy, since they are the major causes of hemiparesis. However said conditions also comprise multiple sclerosis, brain tumors, and other diseases of the nervous system or brain, including schizophrenia or neurodegenerative diseases such as e.g. Parkinson's disease, Alzheimer's disease, spinocerebellar ataxia, Huntington's disease, motor neurone diseases, spinal muscular atrophy, amyotrophic lateral sclerosis, and the like. Impairment of the function of the upper limb and/or hand may also result from surgery, notably from hand surgery. Other conditions which may result

In yet another aspect, the invention provides a method treating upper limb and/or hand impairment in a subject. Said method comprises a first step of diagnosing upper limb and/or hand impairment by the method described above and a second step of providing rehabilitation if said subject has been diagnosed as being impaired in upper limb and/or hand function.

In yet another aspect, the invention provides a method for assessing the efficacy of a treatment of an upper limb and/or hand impairment in a subject in need thereof. According to this aspect of the invention, a subject being treated for upper limb and/or hand impairment is being tested for impairment in upper limb and/or hand function by the method described above. If no such impairment is detected, the treatment is deemed efficacious. If impairment is detected, the treatment is deemed to be lacking in efficacy. Preferably, said treatment is rehabilitation.

In yet another aspect, the invention provides a method for adapting a treatment of an upper limb and/or hand impairment in a subject in need thereof. According to this aspect of the invention, the efficacy of a treatment of an upper limb and/or hand impairment of a subject is first assessed by the method described above. If the treatment is deemed efficacious, no upper limb and/or hand impairment is detected, as explained above, which means that the treatment has reached its goal. In this case, therefore, said treatment may be decreased or stopped. on the other hand, if the treatment is deemed to be not efficacious, i.e., if impairment is detected, said treatment may be continued or increased.

The invention also relates to a product/computer program containing a set of instructions characteristic of the implementation of the inventive method.

The invention also relates to a processing system including a computation unit and an input interface, characterized in that said system includes means for implementing the method for evaluating manual dexterity as disclosed herein.

In reference to FIG. 9, a device (1) according to a particular embodiment of the present invention includes a computation unit (10) capable of following computer instructions and processing data. One such computation unit preferentially includes a microprocessor (110) which can be of any type known in the state of the art. The computation unit (10) also has a storage unit (100) that is capable of receiving a computer program including a set of instructions characteristic of the implementation of the method, and is capable of storing data.

The device (1) also includes an input interface (12) connected to the computation unit (10) enabling a subject, i.e., an operator (O) of the device (1), to enter data to be treated. One such input interface (12) includes any element enabling the entry of such data destined for the computation unit (10) such as a keyboard element optionally associated with a pointing device element. Preferably, the input interface (12) comprises the FFM device, wherein the FFM device is connected to the computation unit (10), thus enabling the performance of a the operator (O) to be directly entered to be treated.

Preferentially, the computation unit further includes an output interface (14) such as a screen that on the one hand enables the user to verify the integrity of the data entered but on the other hand enables the computation unit (10) to be able to interact with the operator (O). In particular, the an output interface (14) enables the computation unit (10) to display instructions, notably visual cues, for the user.

The device (1) can be integrated in a single system such as a computer, a smartphone or any other system known in the state of the art enabling implementation of the inventive method. The operator (O) can be of any skill level and thus may or may not have medical qualifications.

It is notably envisaged according to a particular embodiment of the present invention that the data entered by the operator (O) are sent via a network (the Internet, for example) preferentially in a secure manner to a remote server comprising a computation unit capable of implementing the inventive method and thus of treating the data received by the server. Optionally, after said processing, the server returns the result of the analysis to the user via the same network or another. Optionally, the server records the data and/or the result of the analysis on a means of recording. Obviously, means of guaranteeing the anonymity of the physiological/clinical characteristics of the donor and the recipient can be envisaged.

Thus, one such device (1) enables implementation of the inventive method, i.e., it enables implementation of the following steps:
displaying visual cues on the an output interface (14) such as a screen;
entering performances of the operator in the following tasks using the input interface (12) into a computation unit (10) (step 22), said characteristics including:
   Finger force tracking,
   Single finger tapping,
   Multi finger tapping, and
   optionally Sequential finger tapping;
comparing for each task the performance of the subject with the performance of a healthy subject via data processing by the computation unit (10) (step 23), and
analyzing said comparisons so as to determine manual dexterity (step 24).

Thus, the method of the invention can be implemented not only by clinical or hospital personnel but also by all persons involved in clinical research (pharmaceutical industry, scientists, doctors, etc.).

It is understood that the examples described above in no way serve to limit the true scope of this invention, but rather are presented for illustrative purposes. All publications and patent applications cited in this specification are herein incorporated by reference as if each individual publication or patent application were specifically and individually indicated to be incorporated by reference.

### FIGURE LEGENDS

FIG. 1—the Finger Force Manipulandum (FFM). Index, middle, ring and little finger each apply forces on a spring-loaded piston. Two types of tasks were implemented: continuous force tracking and finger tapping. Forces applied by each finger were recorded via a CED interface (not shown) and used for real-time visual feedback and for performance analysis.

FIG. 2—The four FFM tasks. A-D): Left panels: Setup with FFM and screen providing visuo-motor feedback. Right panels: Example recordings of finger force traces. Index finger: red, middle: blue, ring: green, little: turquoise. The target for each finger is shown as a line of the same color (trapezoid form in A,B,D). Left column: control subject. Right column: stroke patient. A) Finger force tracking. Screen: The yellow line represents the target force and the cursor (here close to the ramp) represents the instantaneous force exerted by the index finger. The subject has to match the vertical cursor position with the target force. Right panels: tracking examples of five subsequent trials. Note: the patient's tracking force trace is more irregular, does not return to baseline between trials and the little finger (turquoise) applies unwanted force (motor overflow). B) Sequential finger tapping: Screen: the height of 4 red vertical bars represents the force exerted by each finger. Next to each finger feedback the target bar (white), here only visible for the index finger. Successively appearing target bars indicate the 5-tap finger sequence (e.g., digit 3-2-4-5-3). Right panels: correct tapping sequence for the control subject, erroneous sequence in the patient. C) Single finger tapping: Screen: ring finger is indicated as tapping finger (white bar). Visual feedback was only provided for the tapping finger (red bar). Right: index finger 1 Hz condition with (15 s) and without (20 s) tapping cue. Less finger taps, incomplete return to baseline and unwanted movements of other fingers are noticeable in the patient. D) Multi-finger tapping: Screen: two-finger target tap (index and ring finger, white bars) and corresponding two-finger user tap (red bars). Right: four subsequent trials, each with a different finger combination (ring-little; little; middle-ring; index). The patient clearly has more difficulties.

FIG. 3—Finger force tracking. Group comparison between control subjects (square) and stroke patients (circle). A) Mean RMSE for index finger tracking (±95% confidence interval) for ramp and hold phase combined. B) Mean release duration for trials at 1N and 2N with the index finger. C) Mean baseline force between trials. Asterisks indicate (here and in the following Figures) significant differences between the two groups, with *p<0.05 and **p<0.01.

FIG. 4—Sequential finger tapping. Group comparison between control subjects (square) and stroke patients (circle). A) Mean success rate across all trials (learning and recall, sequence A, B and C) of the sequential finger tapping task. A success rate of 1 indicates perfect performance. B) Mean number of correct taps (max=5) for the first half (‘1’) and the second half (‘2’) of the learning phase of for each sequence (A, B and C). Note: patients and controls had similar numbers of correct taps at the first half of sequence A, controls subsequently increased their performance significantly (+++). In controls, learning during sequence A improved initial performance in subsequent sequences B and C: they had significantly more correct taps at the first halves of the sequences B and C (B: P=0.04; C: P=0.03) compared to patients. Significant differences between and within groups are indicated.

FIG. 5—Single finger tapping. Group comparison between control subjects (square) and stroke patients (circle). A) Mean tapping rate across all tested digits at 1 Hz, 2 Hz and 3 Hz. B) Mean number of unwanted extra-finger-taps during each condition. C) Mean number of non-wanted overflow taps across all conditions.

FIG. 6—Multi-finger tapping. Group comparison between control subjects (square) and stroke patients (circle). A) Mean success rate for each finger during one- and two-finger taps. B) Mean success rate for each combination of finger(s) to activate (one or two fingers).

FIG. 7—Individual dexterity profiles. A-B) Force tracking, C-D) Single finger tapping, E-F) Multi-finger tapping. A) Index finger force tracking: mean error score for each stroke patient (P01-P10). The ‘normality threshold’ (control average+25D) is indicated by a horizontal line (and its corresponding value). Individual scores>threshold were considered pathological. B) Index finger force tracking: mean release duration. C) Single finger tapping rate: 1 minus the slope 1-3 Hz value for each patient. D) Single finger tapping: number of overflow taps during the 1 Hz condition. E) Multi-finger tapping: omission rate across all trials. F) Multi-finger tapping: number of unwanted extra-finger-taps (UEFTs) for one-finger combination trials. Patient P01 did not perform this task.

FIG. 8—Correlations with clinical scores. A-B) FFM single finger tapping (N=10): A) Correlation between 1-3 Hz slope and the ARAT scores. B) Correlation between 1-3 Hz slope and the Moberg pick-up scores. C-D) FFM multi-finger tapping (N=9). C) Correlation between success rate and the ARAT scores. D) Correlation between success rate and the Moberg pick-up scores.

FIG. 9—Schematic representation of a processing system according to a particular embodiment of the present invention

FIG. 10—Functional graph representing a method according to a particular embodiment of the present invention

### EXAMPLES

### Method

### Subjects:

Ten adult stroke patients were recruited at the Rehabilitation clinic at Sainte-Anne Hospital, Paris. All patients suffered from a single ischemic or hemorrhagic stroke and were at least 2 weeks post-stroke at the time of their participation to the study. Included patients had varying degrees of hemiplegia affecting the upper limb and the hand. Exclusion criteria included severe loss of sensation of the affected limb, other neurological conditions and other cognitive dysfunction that would interfere with the understanding of the experiment, such as visual deficits or severe neglect. Ten healthy controls subjects, comparable in age, were also recruited. Table 1 lists the demographic and clinical details. The procedures of the study complied with the Declaration of Helsinki, and subjects provided informed consent.

TABLE 1
Clinical measures.

| | | | | | | | | Maximal | | Moberg | Mono- |
|---|---|---|---|---|---|---|---|---|---|---|---|
| | | | | | | | | grip | | Pick-Up | filaments |
| | | | | | | Time | | force (kg) | | Test (s) | (g) |
| | | | | Hemi- | | since | ARAT | affected\ | maximal | affected\ | affected\ |
| Participant | | | Lesion | paretic | | lesion | (max = | non- | grip | non- | non- |
| Patients | Age | Gender | location | side | Etiology | (days) | 57) | affected | force (%) | affected | affected |
| 1 | 76 | F | Right precentral gyrus and | left | H | 36 | 57 | 12\16 | 75 | 16\12 | 0.4\0.4 |
| | | | right lenticular nucleus | | | | | | | | |
| 2 | 49 | M | Left parieto-occipital | right | H | 120 | 57 | 42\39 | 100 | 25\13 | 0.4\0.07 |
| | | | cortex, intra-ventricular | | | | | | | | |
| | | | and corpus callosum | | | | | | | | |
| 3 | 25 | M | Right temporo-parietal | left | H | 330 | 32 | 15/44 | 34 | 60\13 | 0.07\ |
| | | | cortex | | | | | | | | 0.07 |
| 4 | 68 | F | Left fronto-parietal cortex | right | I | 19 | 57 | 11/15 | 73 | 19\14 | 0.07\ |
| | | | | | | | | | | | 0.4 |
| 5 | 46 | M | Right sylvian and subdural | left | I | 165 | 51 | 12/26 | 46 | 50\30 | 0.4\0.4 |
| | | | hematoma | | | | | | | | |
| 6 | 68 | M | Left sylvian | right | I | 315 | 40 | 18/37 | 49 | 60\22 | 0.07\ |
| | | | | | | | | | | | 0.4 |
| 7 | 40 | M | Left thalamus | right | H | 75 | 40 | 6/43 | 14 | 51\21 | 0.07\ |
| | | | | | | | | | | | 0.4 |
| 8 | 64 | M | Left pons | right | I | 40 | 57 | 38/30 | 100 | 32\24 | 0.4\0.4 |
| 9 | 50 | F | Left precentral cortex and | right | I | 210 | 56 | 19/24 | 79 | 13\12 | 0.4\0.4 |
| | | | left semi-oval center | | | | | | | | |
| 10 | 65 | M | Left pons | right | I | 180 | 57 | 17/39 | 43 | 26\17 | 0.4\0.4 |
| Patients | 55.1 | 3F/7M | | | | 149 | 50.4 | 31.3(±10.7)\ | 61.3 | 35.2 | 0.33 |
| Mean | (±15.7) | | | | | (±112) | (±9.4) | 19(±11.7) | (±28.5) | (±18.3)\ | (±0.14)\ |
| (±SD) | | | | | | | | | | 17.8 | 0.27 |
| | | | | | | | | | | (±6.2) | (±0.18) |
| Controls | 52.9 | 4F/6M | | | | | | 35.1 ± | | 14.3 | 0.14 |
| Mean | (±17.4) | | | | | | | 11.4 | | (±1.9) | (±0.14) |
| (±SD) | | | | | | | | | | | |

For each stroke patient is indicated: age, gender, lesion location, hemiparetic side, etiology (type of stroke: H=hemorrhagic; 1=ischemic), time since lesion (days), total ARAT (Action Research Arm Test) score, MVC grip force in kg in the hemiparetic and non-affected hand, % MVC in the hemiparetic handcompared to the non-paretic hand, performance of the Mobergpick-up Test (time in s) for both hands, % of proprioception, and tactile sensibility (Semmes-Weinstein mono-filament test) for both hands. Bottom two lines: mean and standard deviation in stroke patients and control subjects.

### Clinical Measures:

The Arm Research Action Test (ARAT), a clinical test for grasp, grip, pinch and gross movement in the hemiparetic hand, was used as a global measure of hand function [33,34]. The Moberg pick-up test was used as a clinical assessment of manual grip function in each hand. Time taken to place all 12 objects into the box was recorded. The time taken reflects the degree of precision grip function (>18 seconds is considered pathological in this age span) [35]. A Semmes-Weinstein mono-filament test with three calibers (2 g, 0.4 g and 0.07 g) was used to measure the tactile sensitivity of finger tips in each hand [36]. Maximal grip force (in Kg) in each hand was recorded with a hydraulic Jamar dynamometer (http://www.kinetec-byvivadia.com). Proprioception was tested by assessing the subjects' capacity to detect and match passive finger displacement in one hand while keeping the eyes shut and rated as intact, impaired or absent. All measures were also obtained in control subjects, except the ARAT.

### Finger Force Manipulandum (FFM):

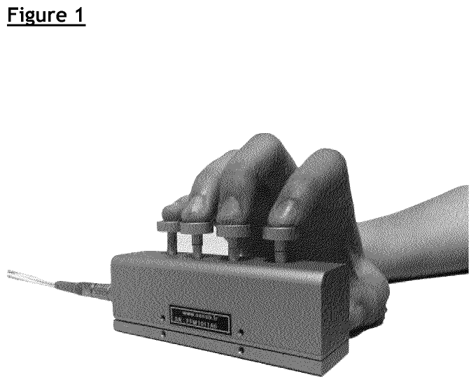

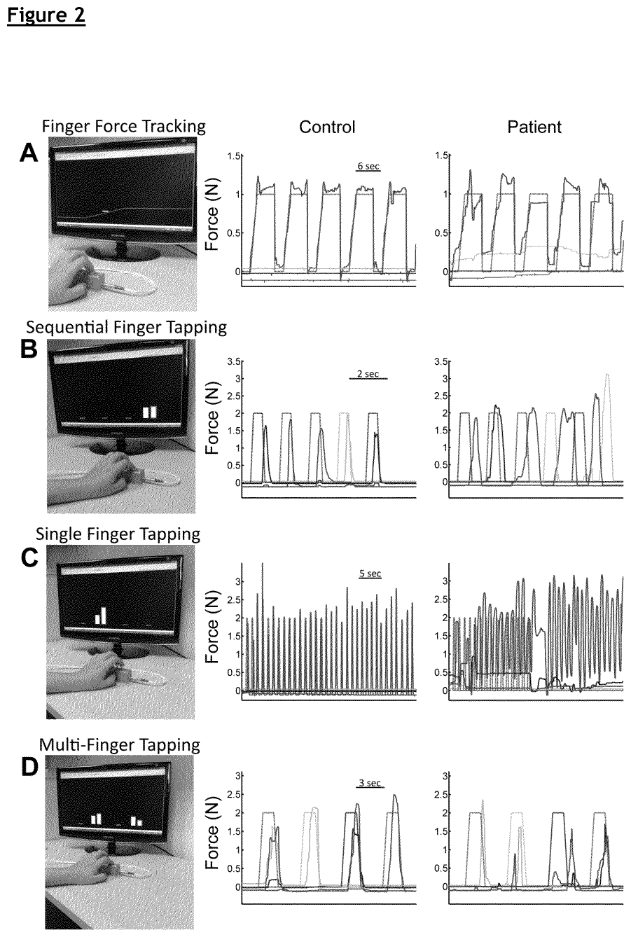

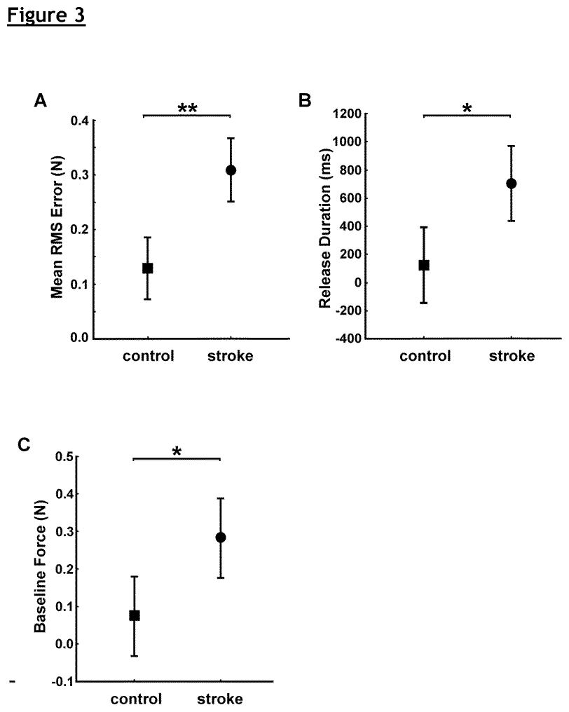

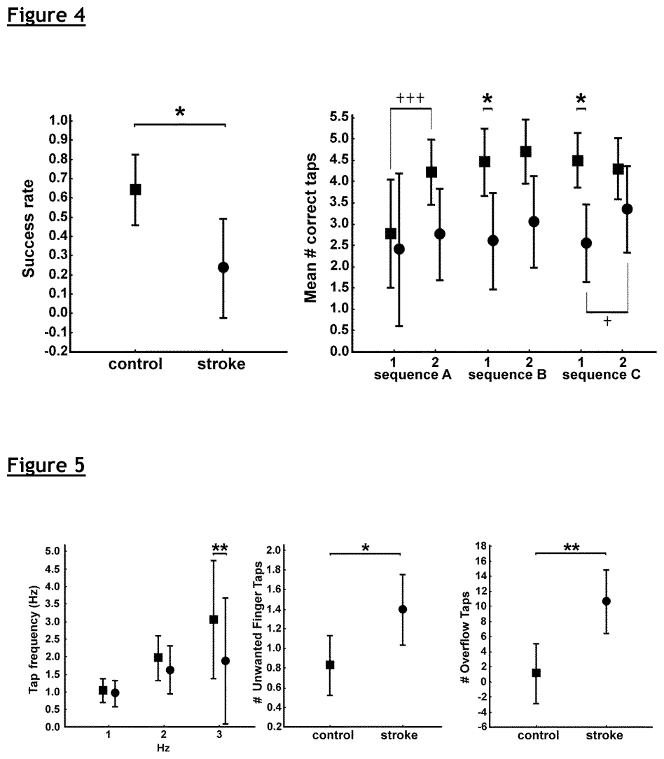

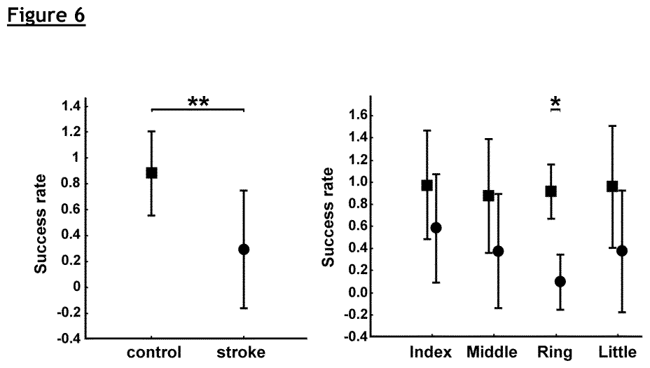

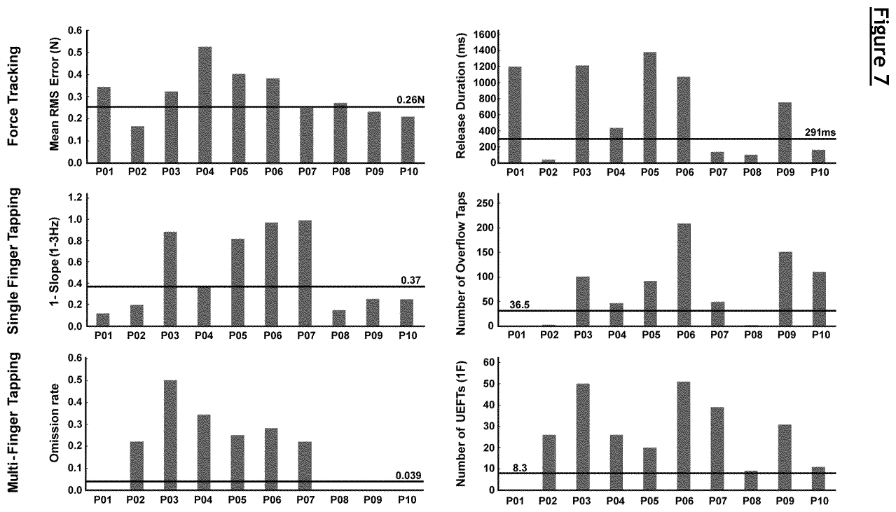

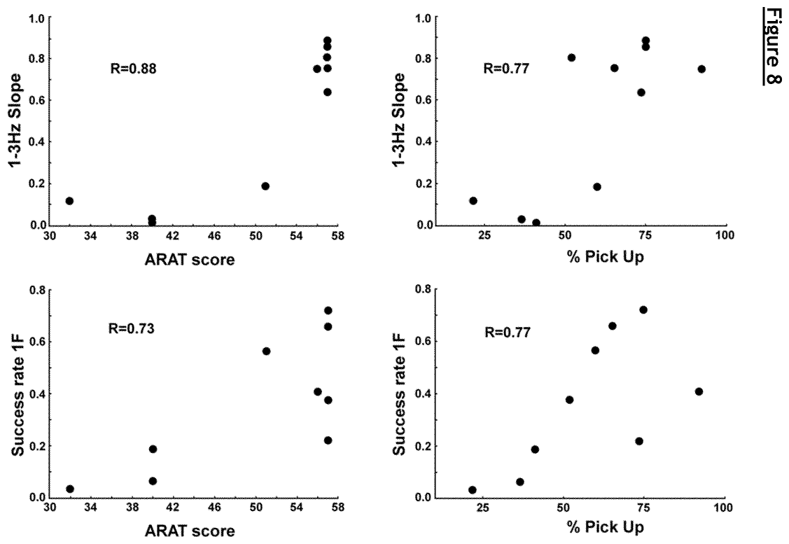

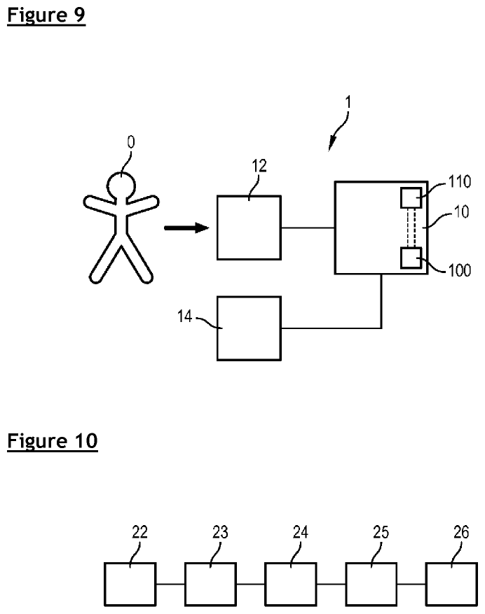

Together with Sensix (www.sensix.fr) we developed the Finger Force Manipulandum (FFM) in order to quantify key components of manual dexterity in stroke (and other) patients. The FFM is equipped with four pistons positioned under the tip of the index, middle, ring and little finger, each coupled to an individual strain gauge force sensor (FIG. 1). With increasing force the pistons move against a spring load over a range of 10 mm. The end of this dynamic (non-static) range is reached with 1N. Above 1N, forces are controlled isometrically. Thus each sensor measures force along the piston axis exerted from each finger independently. The precision of the sensor is <0.01N, with a range of 0-9N. Force data of each finger was sampled to a CED 1401 (with 10 kHz sampling rate/digit) connected to a computer running Spike 2v6 (Cambridge Electronic Design, www.ced.co.uk) software. Custom-written CED scripts provided real-time visual display of digit forces and target instructions or target forces.

### FFM Tasks:

Four separate tasks (i-iv) were developed in order to quantify different components of manual dexterity. In all tasks the subject was first required to place the fingers on the pistons and was instructed to maintain the fingers on the pistons throughout the tasks. Every subject was able to use the FFM with the forearm supported on the table. To ensure a comfortable position some subjects used a silicone wrist support during the tasks.

(i) The Finger Force-Tracking task is a visuo-motor task of finger force control. By varying the force on the piston with the finger, the subject controlled a cursor on a computer screen (FIG. 2A). The subject was instructed to follow the target force as closely as possible with the cursor. The target force (a line) passed from right to left over the screen, presenting successive trials. Each trial consisted of a ramp phase (a linear increase of force over a 1.5 s period), a hold phase (a stable force for 4 s) and a release phase (an instantaneous return to the resting force level, 0N) followed by a resting phase (2 s). Trials were repeated 24 times, distributed in four blocks of 6 trials, two blocks with a target force of 1N and two with a target force of 2N. In this study, patients performed the finger force-tracking task separately with the index and the middle finger of their hemiparetic hand and controls performed the task with their index and middle finger of their right hand. Task duration was 3 min 20 s/digit.

(ii) The Sequential finger tapping task is a 5-tap finger sequence involving the four digits. The visual display consisted of 4 columns (representing the 4 digits), whose height varied in real-time as a function of exerted finger force (feedback). In addition, a target column (cue) adjacent to each feedback column indicated the piston to be pressed (FIG. 2B). The subject was instructed to press the indicated piston as soon as the target appeared. Each sequence was repeated 10 times with visual cues (learning phase) and then repeated 5 times from memory, i.e. without cues, and as quickly as possible (recall phase). Force feedback was always present. In this protocol, the subjects performed three previously unknown motor sequences: they first learned and then repeated the sequence (A) 2-5-3-4-2 (2=index; 5=little); then the sequence (B) 4-3-5-2-4 and finally the sequence (C) 3-2-4-5-3. A single sequence (trial) of 5 taps lasted 5 s and the duration for all 15 trials was 2 min 20 s.

(iii) The Single finger tapping task consisted of repetitive tapping with one finger with or without an auditory and simultaneous visual cue. The visual display was similar to that in task (ii). Three tapping rates were tested: 1, 2 and 3 Hz (similar to [9]). After the cued tapping period (15 taps) the subject was required to continue tapping for a similar period, without cue but at the same rate. The subject started at 1 Hz with the index finger, followed by the middle (FIG. 2C), ring and little finger. This protocol was repeated at 2 Hz and then at 3 Hz. The total duration of this task was 4 min.

(iv) The Multi-finger tapping task consisted of simultaneous tapping with different finger configurations in response to visual instructions. The visual display was similar to that in task (ii) and (iii). Subjects were instructed to reproduce 11 different finger tap configurations following the visual cue (FIG. 2D). The 11 different configurations consisted of 4 single-finger taps (separate tap of index, middle, ring or little finger), 6 two-finger configurations (simultaneous index-middle, index-ring, index-little, middle-ring, middle-little or ring-little finger taps), and one four-finger tap. All configurations were performed twice resulting in a total of 32 (4×8) single-finger taps, 30 (6×5) two-finger taps and 2 four-finger taps. Performance measures were calculated for single and two-finger configurations. Four finger taps were not analyzed. The order of the configurations was pseudo-randomized with equal number of transitions between one and two-finger taps. The entire task with its 64 trials lasted 4 mins 40 s.

### Data Analysis:

Task performance was analyzed using Matlab (v7.5, The MathWorks, Inc., Natick, Mass., USA). The four force signals were first down-sampled to 100 Hz for the analysis.

Finger force tracking: all performance measures were calculated trial-by-trial (N=24). Tracking error was calculated as the root-mean-square error (RMSE) between the actual applied force and the target force. The error was separately extracted during the ramp and the hold phase. The time of the force onset in response to the target ramp and the time of the release onset at the end of the hold phase were calculated as threshold crossings of dF/dt. The release duration was computed as the time taken to reduce the force from 75% to 25% of the target force. The coefficient of variation (CV) of force (i.e. SD/mean) was calculated separately for the ramp and the hold phase. Mean force during the hold was calculated as the average force across 3 s excluding the first and last 500 ms of the hold phase. Mean baseline force was calculated as the average force during the resting phase between each trial from 1500 ms to 500 ms before the ramp onset.

For the three tapping tasks the finger taps were identified in a similar way. Starting from the force trace each tap was identified as a discrete event according to threshold (>0.5N) allowing identification of target and the applied force peaks (retained as taps). The time location and amplitude of each tap were then recorded. The following task-specific performance variables were then obtained:

In the Sequential finger tapping task we computed the number of user taps trial-by-trial, i.e. for each 5-tap target sequence. By comparing the user taps to the target sequence, each trial was then labeled as correct or incorrect. In case of an incorrect sequence the number of missing or additional unwanted taps was recorded, as well as the number of consecutive correct taps within parts of the sequence. Furthermore, performance was calculated across trials, by computing the number of correct trials and the number of error taps for each finger. These measures were obtained for the learning and the recall phase, respectively.

In the single finger tapping task the lead-finger (target finger) and the non-lead-fingers were identified in each condition (finger and 1, 2 or 3 Hz). For the lead-finger the number of taps, the tap amplitude, and the interval between consecutive taps were calculated for each condition. Unwanted taps were identified in the non-lead-fingers and labeled as overflow taps (non-lead-finger tap at the same time as a lead-finger tap) or as unwanted finger taps (non-lead-finger tap in the absence of a lead-finger tap). To estimate the capability to adapt the tapping speed to the target frequency of the cue we calculated the slope of the tapping speed across the 1 Hz, 2 Hz and 3 Hz conditions in the index finger. A slope=1 indicates correct tapping speed, a slope<1 slower execution.

In the multi-finger tapping task each tap, in response to a displayed finger configuration, was identified as correct or incorrect, i.e. identical to or different from the required target taps. Errors, in each finger, were categorized as missing taps (omissions, omission rate), or as unwanted extra-finger-taps (one or several) (errors reported in keyboard typing [37]). Across trials the number of errors was evaluated as a function of the target (one- or two-) finger configuration.

Finally, in order to obtain individual profiles of dexterity components, we plotted each patient's performance in three of the four tasks and compared it to the performance range observed in the control group. This was done for six performance measures which were found to differ between groups (i.e., considered as discriminative variables). Values beyond the control group's mean+2SD in a given measure were considered pathological.

### Statistical Analysis:

Descriptive statistics are shown as mean±SD. Student's T-test was used to test for group differences in single-level variables. Differences in the measures obtained from the four tasks described above were tested using repeated measures ANOVAs. (i) Force tracking: independent variables (error, timing, etc.) where studied with ANOVA including one between-group factor GROUP (patients, controls), and three within-subject levels: FINGER (index, middle), FORCE (1N, 2N), PHASE (Ramp, Hold). (ii) Sequential finger tapping: independent variables (success rate, number of correct taps) where studied with ANOVA including one between-group factor GROUP (patients, controls), and three within-subject levels: SEQUENCE (sequence A, B, C), PHASE (learning and recall phase), TRIALS. (iii) Single finger tapping: independent variables (tap frequency, number of overflow taps, etc.) where studied with ANOVA including one between-group factor GROUP (patients, controls), and three within-subject levels: FREQUENCY (1, 2, 3 Hz), FINGER (index, middle, ring, little) and PHASE (with auditory cue, without auditory cue). (iv) Multi-finger tapping: independent variables (success rate, number of unwanted extra finger taps, etc.) where studied with ANOVA including one between-group factor GROUP (patients, controls) and one within-subject levels: TRIALS. Post-hoc tests were performed using Fisher LSD Test. Spearman's rank order correlation was used to investigate correlations between performance measures and clinical scores. Jamar and Moberg Pick up scores were presented as % of non-hemiparetic hand scores for correlation tests. Pearson's correlation was used to test for relations between different performance measures. The level of significance was set to p<0.05.

### Results

### Clinical Assessment of Hand and Finger Function

In stroke patients maximal power grip force in the paretic hand was significantly reduced to a mean of 19 kg compared to 35 kg in controls (P<0.01). According to the ARAT, none of the patients were severely impaired (score<5), five patients had moderately impaired hand function (51<score<57), and another five scored the maximal 57 points [38]. However, three of these latter patients had reduced maximal grip force and all four were slower in the pick-up test with the affected hand (Table 1). Sensory thresholds in the fingers were also significantly decreased in stroke patients (P<0.05) but only patient 3 had impaired proprioception (Table 1).

### Task Feasibility

All ten patients were able to accomplish the force tracking task and the single finger tapping tasks, and nine patients completed the multi finger tapping task. However, only four of ten patients achieved the sequential tapping task. The main issues affecting feasibility were: maintaining all four fingers on the pistons and the sequential taping task being too fast (Table 2).

TABLE 2
FFM ergonomic and task feasibility in hemiparetic patients.

| | Ergonomic difficulties with the FFM | | | | | | | |
|---|---|---|---|---|---|---|---|---|
| | device | | | Task feasibility | | | | |
| | | | Interaction | Finger | Sequential | Single | | |
| | Arm | Finger | with | Force | finger | finger | Multi- | Problem |
| Patients | posture | position | computer | Tracking | tapping | tapping | fingertapping | encountered |
| 1 | no | Maintaining | Difficulties | yes | no | yes | no | Too fast |
| | | little finger on | to | | | | | and |
| | | piston (short | interact | | | | | difficult |
| | | little finger) | with the | | | | | (sequence) |
| | | | computer | | | | | Failed to |
| | | | feedback | | | | | use |
| | | | | | | | | computer |
| | | | | | | | | feedback |
| | | | | | | | | (sequence |
| | | | | | | | | and |
| | | | | | | | | tapping) |
| 2 | no | no | no | yes | yes | yes | yes | / |
| 3 | Maintaining | Fingers slide | no | yes | no | yes | yes | Too fast |
| | wrist | on pistons | | | | | | and |
| | extension | (flexor | | | | | | difficult |
| | (flexor | spasticity) | | | | | | (sequence) |
| | spasticity) | | | | | | | |
| 4 | no | Maintaining | no | yes | no | yes | yes | Too fast |
| | | little finger on | | | | | | and |
| | | piston (short | | | | | | difficult |
| | | little finger) | | | | | | (sequence) |
| 5 | no | no | Difficulties | yes | no | yes | yes | Too fast |
| | | | to | | | | | and |
| | | | interact | | | | | difficult |
| | | | with the | | | | | (sequence) |
| | | | computer | | | | | Failed to |
| | | | feedback | | | | | use |
| | | | | | | | | computer |
| | | | | | | | | feedback |
| | | | | | | | | (sequence) |
| 6 | Maintaining | Maintaining | no | yes | no | yes | yes | Too fast |
| | wrist | fingers on | | | | | | and |
| | extension | pistons | | | | | | difficult |
| | (weak | (adductor | | | | | | (sequence) |
| | extensor) | spasticity) | | | | | | |
| 7 | no | Maintaining | no | yes | no | yes | yes | Too fast |
| | | little finger on | | | | | | and |
| | | piston | | | | | | difficult |
| | | (contracture | | | | | | (sequence) |
| | | of little | | | | | | |
| | | finger) | | | | | | |
| 8 | no | Maintaining | no | yes | yes | yes | yes | / |
| | | fingers on | | | | | | |
| | | pistons | | | | | | |
| | | (repositioning) | | | | | | |
| 9 | no | no | no | yes | yes | yes | yes | / |
| 10 | no | no | no | yes | yes | yes | yes | / |
| Feasibility | 8/10 | 4/10 | 8/10 | 10/10 | 4/10 | 10/10 | 9/10 | / |

Indicated are for each patient: qualitative observations in terms of ergonomic feasibility and task feasibility.

### Force Tracking

Patients and controls applied the same amount of force during the hold phase in 1N (controls: 0.98N±0.2; patients: 1.1N±0.2; P=0.24) and 2N conditions (controls: 1.9N±0.4; patients: 2.0N±0.2; P=0.36). This task revealed dramatic differences in the precision of force control: stroke patients showed increased tracking error (0.31N±0.1) compared to controls (0.13N±0.06). This difference was highly significant (GROUP effect: F=21.18; P<0.001; FIG. 3A) and was apparent during both the ramp and hold phases, and at both force levels (P<0.05). Performance was equally impaired when using the index or the middle finger. Furthermore, time taken to release force at the end of the hold period (FIG. 3B) was significantly prolonged (about seven times longer) in stroke patients (702 ms±557) compared to controls (123 ms±84)(GROUP effect: F=5.03; P=0.014). Patients also showed difficulty in not applying force (relaxing) with the lead-finger during the baseline (i.e. between trials, see FIG. 2A). The mean baseline force (FIG. 3C) was significantly different and about three times higher in patients (0.28N±0.21) compared to controls (0.07N±0.09; GROUP effect: F=4.10; P=0.028).

Some measures did not reveal any significant difference between groups: this was the case for the timing of the force onset (prior to the ramp) and for the release onset (at the end of the hold phase). Also the CV of tracking force was similar in the two groups.

### Sequential Finger Tapping

The sequential finger tapping task turned out to be relatively difficult. Control subjects achieved a grand average success rate of 0.66±0.2, measured across all trials of the two conditions (learning and recall phases) and across the three different sequences (A, B, C). The four patients that accomplished this task reached a significantly lower success rate of 0.23±0.28 (FIG. 4A, GROUP effect: F=8.21; P=0.017). Both groups showed similar performance in the first half of sequence A (FIG. 4B). During the learning phase (i.e. the cued condition), controls improved their performance by passing from a mean number of 2.7 (/5) correct taps to 4.2 (/5) between the first half and the second half of the learning phase for sequence A (P<0.001; FIG. 4B). Controls showed maintained performance without obvious learning for the subsequent sequences B and C. In the patients significant improvement of performance between the first and the second half of the learning phase was only seen during the last sequence (sequence C): they passed from 2.5 (/5) correct taps to 3.4 (/5) (P=0.02; FIG. 4B). In patients, no improvement was apparent during the first two sequences A and B. A significant improvement occurred only between the first and second halves in sequence C. No group differences were found in second halves of each sequence (FIG. 4B) nor in the recall phases.

### Single Finger Tapping

We measured the average single finger tapping rate, cumulated over the cued and the non-cued condition (FIG. 5A). Controls were able to follow the imposed tapping rate, with a mean rate of 1.06 Hz±0.06, 1.98 Hz±0.13 and 3.17 Hz±0.47 for the 1, 2 and 3 Hz condition, respectively. The tapping rate was impaired in patients, with a reduced tapping rate of 2.31 Hz±0.69 at the 3 Hz condition compared to controls (GROUP*FREQUENCY effect: F=9.30; P<0.001; post-hoc GROUP effect at 3 Hz: P<0.001; but not at 1 or 2 Hz). Thus, patients had a decreased slope of tapping rate (1 Hz-3 Hz) at 0.5±0.37 compared to controls (1.06±0.22; T=−4.12; P<0.001). There was no difference in tapping rate between the cued and non-cued condition. No difference between groups was found in the tapping regularity with no significant difference for the mean tap interval.

Unwanted finger taps occurred rarely during single finger tapping, i.e. a tap of a non-lead finger in the absence of a lead-finger tap. Per condition (Frequency/Finger: 35 taps) this occurred on average 0.8 times (0.8 taps/35) in controls, but significantly more often (1.4 taps/35) in patients (FIG. 5B, GROUP effect: F=6.60; P=0.021).

In patients the single finger tapping task also produced substantial unwanted motor overflow to fingers not involved in the task (i.e., non-lead finger taps concomitant with lead-finger taps). Patients showed significantly more overflow taps than controls (FIG. 5C, GROUP effect: F=12.16; P=0.003). At 1 Hz patients made on average 10 extra overflow taps per condition (frequency/finger: for a total of 35 required taps) compared to a single overflow tap in controls. In both groups overflow taps were least frequent when the index or little finger acted as lead finger.

### Multi-Finger Tapping Task

We first computed the grand average success rate across single- and two-finger combinations. Patients with a mean success rate of 0.3 were less accurate compared to control subjects with a mean success rate of 0.9 (FIG. 6A, GROUP effect: P<0.001). This group difference was present in both one- and two-finger combinations (P<0.05).

For one-finger taps, a FINGER×GROUP interaction was found (FIG. 6B, FINGER×GROUP effect: F=5.90; P=0.002). Posthoc testing showed significantly lower success rate in the ring finger in patients (with a success rate close to 0.1 for patients compared to 0.9 for controls; P<0.05). For each failed one or two-finger trial, we computed two types of errors. The grand average omission rate was significantly greater in patients 0.2±0.17 compared to controls 0.01±0.01 (T=3.31; P=0.01). Summed across trials and fingers, unwanted extra-finger-taps were more frequent in patients (54±24.1) than in controls (7.9±6.9; T=5.52; P=0.0003).

The distribution of unwanted extra-finger-taps across fingers is shown in Table 3 for both one and two-finger combinations. Each line in the Table shows the occurrence of unwanted extra-finger-taps as a function of finger combination. For every target combination, patients produced more error in other fingers than control subjects. In the least successful one-finger combination (the ring finger target tap) patients erroneously activated also the middle finger in more than sixty percent of the trials, while this was the case in less than ten percent in controls (Table 3). Note that the index and little finger also made errors in this condition, but less frequently (in about 35%) than the middle finger. This same error pattern across fingers (i.e. middle finger error>index or little finger error) was also present in control subjects, but in an attenuated form. More generally, the pattern of unwanted extra-finger-taps formed a ‘neighborhood’ gradient, such that digits anatomically far from the target (lead) digit produced less error taps than those closer to (or immediate neighbors of) the target digit. This also held for the ‘2-3’ and ‘4-5’ two-finger combinations. Two-finger combination taps of non-adjacent digits (‘2-4’, ‘2-5’, ‘3-5’), showed, in absence of a distance gradient, a balanced error distribution. Similar but attenuated ‘across’ finger error patterns were also observed for the control subjects.

TABLE 3
Finger tap errors as a function of target tap combination

| | | Stroke patients | | | | Controls | | | | |
|---|---|---|---|---|---|---|---|---|---|---|
| | | Digit 2 | Digit 3 | Digit 4 | Digit 5 | Digit 2 | Digit 3 | Digit 4 | Digit 5 | |
| one-finger tap | 2 | X | 33% ± 34 | 24% ± 31 | 10% ± 8 | X | 1% ± 4 | 0% | 0% | |
| (target digit) | 3 | 42% ± 27 | X | 28% ± 25 | 9% ± 12 | 4% ± 8 | X | 8% ± 12 | 1% ± 4 | =0% |
| | 4 | 35% ± 29 | 65% ± 32 | X | 36% ± 36 | 0% | 6% ± 9 | X | 3% ± 8 | <20% |
| | 5 | 18% ± 21 | 27% ± 31 | 33% ± 31 | X | 0% | 0% | 4% ± 8 | X | <40% |
| two-finger taps | 2-3 | X | X | 38% ± 38 | 10% ± 14 | X | X | 2% ± 6 | 0% | <60% |
| (target digit) | 2-4 | X | 47% ± 35 | X | 40% ± 33 | X | 18% ± 22 | X | 8% ± 14 | >60% |
| | 2-5 | X | 38% ± 41 | 44% ± 28 | X | X | 2% ± 6 | 8% ± 19 | X | |
| | 3-4 | 53% ± 35 | X | X | 29% ± 28 | 4% ± 13 | X | X | 0% | |
| | 3-5 | 44% ± 30 | X | 58% ± 38 | X | 16% ± 30 | X | 48% ± 37 | X | |
| | 4-5 | 31% ± 30 | 62% ± 38 | X | X | 0% | 10% ± 14 | X | X | |

Total error: finger force tracking error;
RD: release duration;
OF 1 Hz: number of overflow taps in 1 Hz condition;
UEFT 1F: number of unwanted finger taps during one-finger conditions.
Grey shaded correlations: significant at p < 0.05.

Each line shows the occurrence of error taps during multi finger tapping. Error occurrence is given for each finger in % (mean±SD) of target taps in the relevant condition for patients (left) and in control subjects (right). Example: in 10% of all one-finger target taps of the index finger (target digit 2), patients also tapped erroneously with the little finger (digit 5). The first four lines describe everyone-finger target tap condition, the following six lines every two-finger target tap combination. “Xs” indicate coincidence of target finger(s) and correct tap finger(s). Color scale indicates the level of error: white=no error (0%), red>60% errors.

### Individual Dexterity Profiles:

Although significant group differences were found in several dexterity components, not all measures were pathological in all patients (above mean+2SD threshold). For example, only 6 (of 10) patients showed pathological tracking error (FIG. 7A). Furthermore, only 3 patients (P03, P05, P06) showed pathological scores in all 6 measures. The presence of a pathological score in one variable did not always coincide with the presence of pathological scores in other measures. Neither did absence of one pathological score indicate absence in all other scores. The most common profile (in 4 patients) was a combination of five affected dexterity components: release duration, tracking error, number of overflow taps, omission rate and unwanted extra-finger-taps. These five components were increased compared to control thresholds.

Relations and Correlations with Clinical Measures

Individual dexterity profiles in patients (as described above) were not completely coherent with clinical scores. Among the five patients with a maximal ARAT score (P01, P02, P04, P08, P10), and therefore considered as having normal gripping and gross-motor hand function, all were affected in at least one of the six measures. Four different profiles were observed: P04 had pathological scores in all six FFM measure. P10 had pathological scores in three measures: two in the multi-finger tapping task and one in the single finger tapping task (high number of overflow taps). P02 and P08 had pathological scores for two scores of the multi-finger tapping task, but not in the other tasks. Finally, P01 had pathological performance in the two measures of the force tracking task only.

We tested for correlations between the obtained performance measures in the FFM tasks and the ARAT or the Moberg pick-up test scores. Single finger tapping 1-3 Hz slope appeared to be correlated with the ARAT score (FIG. 8A, R=0.88; P<0.001) and with % Pick Up scores (FIG. 8B, R=0.77; P=0.004). The higher the slope during the single finger tapping task, the better were their ARAT or Pick Up scores. Multi-finger tapping success rate also appeared to be correlated with the ARAT score (FIG. 8C, R=0.73; P=0.03) and with % Pick Up (FIG. 8D, R=0.77; P=0.02). Again, a higher success rate in the multi-finger tapping task was found in patients with higher ARAT or % Pick Up scores. For the Finger force tracking task we did not find any correlations between performance variables and clinical measures. We also tested the inter-relations between the 6 measures used for the description of the dexterity profiles and we found four significant correlations among the 15 comparisons (Table 4). The strongest correlation was between 1-3 Hz slope and the unwanted extra-finger-taps (1F) (R²=0.55).

TABLE 4
Pearson correlation coefficients (R2) between dexterity component scores.

| | | Finger | | | | | |
|---|---|---|---|---|---|---|---|
| | | force tracking | | Single finger tapping | | Multi-finger tapping | |
| | | Total error | RD | 1-slope (1-3 Hz) | OF 1 Hz | Omission rate | UEFT 1F |
| Finger force | Total error | | | | | | |
| tracking | RD | 0.38 | | | | | |
| Single finger | 1-slope (1-3 Hz) | 0.28 | 0.19 | | | | |
| tapping | OF 1 Hz | 0.10 | 0.11 | 0.27 | | | |
| Multi-finger | Omission rate | 0.49 | 0.14 | 0.47 | 0.04 | | |
| tapping | UEFT 1F | 0.21 | 0.24 | 0.55 | 0.27 | 0.47 | |

### DISCUSSION

We developed a novel device to quantify manual dexterity in a clinical context. We demonstrated that this device (the ‘FFM’) allows for extraction and quantification of key control variables of manual dexterity in healthy subjects and in stroke patients. The patients tested in this study were able to use the FFM and performed most of the tasks suggesting adequate feasibility of the new method. Performance was impaired in all four visuo-motor tasks: patients showed less accurate force control, slowed finger tapping rate, more error in finger selection and in sequential finger tapping. We also found that patients were not equally affected across different components of manual dexterity which suggests the presence of individual dexterity profiles. These findings will be discussed in turn below.

### Feasibility

Healthy subjects had no problems performing the tasks and our mild-to-moderately affected hemiparetic patients were able to accomplish three out of the four visuo-motor tasks. They encountered, however, difficulties in the sequential finger tapping task, presumably due to an inadequate (too high) task velocity. In terms of ergonomics, patients sometimes encountered problems in maintaining their fingers on the pistons, mostly for the little finger. This led some patients to look at their fingers, rather than at the screen, in order to replace them on the pistons. This problem could in part be due to decreased tactile sensitivity, shown by the Semmes-Weinstein test. The FFM allowed identification of decreased performance in at least one dexterity component in all patients (FIG. 7). Even in patients with maximal ARAT scores (N=5) and in patients with normal Moberg Pick-up times (<18 s, N=2) the FFM revealed deficient manual dexterity components which is coherent with Lang et al, 2006. Although preliminary, given the small sample size, this suggests that the FFM may be more sensitive than other clinical measures in detecting underlying impairments important for dexterity in patients after stroke.

### Task Performance: Group Differences Between Healthy Subjects and Hemiparetic Patients

For the tracking task, which requires explicit control of force, we found increased finger tracking error and longer release duration in patients, consistent with previous reports on power grip force control [18,40]. Patients did not show higher force variability (CV of force) as previously reported [40]. However, this agrees with findings that did not show increased CV when stroke patient performed power grip force tracking at similar absolute forces as the controls [12].

The sequential finger tapping task, which requires motor learning of sequential digit selection, was too difficult for most patients. However, four patients were able to complete the task, but their performance was found to be weaker than in controls. While controls improved their success rate during the first sequence (sequence A) patients improved later in sequence C (FIG. 4B). This is consistent with studies showing intact but slowed motor learning capacity after stroke [27,41].

The single finger tapping task, which requires explicit control of timing, revealed good temporal matching in patients for the 1 Hz and 2 Hz target frequencies, but a reduced tapping rate for the 3 Hz condition compared to controls. Other studies have also shown a decreased maximal finger tapping rate (and decreased regularity) in stroke patients [24,26]. However, we did not find a decreased tapping regularity in patients: this could be due to differences in lesion localizations, e.g. absence of cerebellar lesions in our sample.

During multi-finger tapping, which requires on-line digit selection, patients were less accurate during one-finger or two-finger target taps (made more omissions and unwanted extra-finger-taps). The observed ‘neighborhood’ gradient of unwanted extra-finger-taps in control subjects is consistent with the known degree of independence of finger movements [42] and finger forces [43]. The higher unwanted extra-finger-taps in patients, while the error pattern still followed the distance gradient, reflects decreased finger independence after stoke, consistent with previous reports [10, 22, 44]. Complementary to these previous observations based on purely kinematic measures, we show here that graded finger independence and its impairment in stroke also occurs in a task combining kinetic and kinematic constraints.

Together these findings show that the FFM allows quantification of different key parameters of dexterity with one and the same apparatus in a single one-hour session. The observed impairments of these key parameters in stroke patients with mild-to-moderate hemiparesis were partly consistent with those previously reported in other studies, which confirms the relevance of these measures. Therefore, the FFM allows for a more complete and more sensitive assessment of manual dexterity than previous devices or clinical scores.

### Clinical Correlations

Some of our measures showed correlations with clinical scales. These are, however, to be taken with caution due to limited group size. Nonetheless, these correlations suggest that single finger tapping rate as well success rate in multi-finger tapping correlated with hand functioning according to the ARAT, even though the ARAT showed a ceiling effect. These same two dexterity components also correlated with the Moberg pick-up score. This might point to common underlying control parameters, in particular timing (speed of execution) and digit selection (contrary to Raghavan et al, who did not find any correlations between finger independence indices and clinical scores [22]). The FFM thus provides some measures that correlate with clinical scales, such as the maximal thumb-index tapping rate [24] or the maximal force ratio [17]. This, however, needs to be confirmed in a larger sample size and with a larger variety of clinical scores. The FFM provides a more detailed description of manual dexterity components, but whether these components are independent of each other and how they contribute to explaining variance in hand functioning needs further study.

### Individual Dexterity Profiles

Since the FFM allows for assessment of several different key control parameters it also provides the potential for obtaining individual profiles of impaired dexterity. First, these dexterity profiles varied in the patient group which may reflect a more accurate description of the individual clinical impairment after stroke. Second, these profiles disclosed that various aspects of dexterity might be impaired in a given individual. Clearly, in a given patient some key parameters were impaired while others were not. For example, patient 09 had difficulty in releasing force, avoiding engaging other fingers during taps (overflow and errors) but showed similar accuracy in force tracking and timing compared to controls. This patient therefore had difficulties in stopping and inhibiting movements in other fingers and would likely benefit from targeted training of these components.

The individual profiles (in FIG. 7) suggest that some of the measures are independent of each other, even if the omission rate and the capacity to accelerate the tapping rate seemed to be linked to other measures (Table 4). This, however, will need further statistical elaboration in larger samples. Profiling of impairment should allow extraction of the most severely affected component(s) of dexterity and should permit individual optimization of rehabilitation protocols [45].

### Independence of Finger Movements and Dexterity

In our view, independence of finger movements represents one functional aspect of dexterity, but does not by itself encompass all aspects of manual function. Four different FFM measures allow for characterization of the degree of finger independence. (i) The number of unwanted taps during single finger tapping, and during multi finger tapping, (ii) the success rate, (iii) the omission rate, and (iv) the distribution of unwanted extra-finger-movements. These four measures were impaired in our stroke patients, reflecting a reduced degree of finger individuation.

However, single finger tapping is less complex than multi finger tapping: the latter requires various patterns of instantaneous effector selection. Indeed, the number of unwanted extra-finger-movements during multi-finger tapping was the most affected measure. This deficit in effector selection might be due to non-selective excitation and/or insufficient inhibition [9].

Raghavan et al [22] distinguished the individuation index (a measure of how well the instructed digit moves independently, i.e. without the other fingers moving concurrently) from the stationarity index (a measure of how well a finger remains motionless when another finger was instructed to move). Our multi-finger tapping task provides two corresponding measures: the success rate represents an individuation index, whereas the number of unwanted extra-finger-taps corresponds to a stationarity index. These two indices were negatively correlated with each other (for the index finger: R=−0.91, middle: R=−0.81, ring: R=−0.78, little: R=−0.69, all P<0.001). This indicates that the more a subject had difficulties in moving the instructed finger independently, the more difficulties in keeping this same finger motionless, when other fingers were instructed to move. Such a correlation between individuation and stationarity index was not found by Raghavan et al [22]. Our finding of a relation between finger selectivity and inhibition fits the idea that a general level of tonic inhibition (preventing unwanted movements) is reduced when initiating a movement [46].

Furthermore, the distribution of unwanted extra-finger-taps (in single and two-finger taps) provided two additional insights into how independent finger movements are affected after stroke (Table 3). First, the ring finger was the least independent finger, replicating results from previous studies [9,22]. Second, stroke patients had a similar ‘neighborhood’ gradient as control subjects, suggesting that stroke lesions do not affect this gradient and do not provoke finger-specific deficits (in this stroke group).

Independence of finger movements is not typically a clinical index. Previous studies on independence of finger movements in hemiparetic patients [22,44], all based on kinematics measures, found small or no correlations with clinical hand function scales. Nevertheless, our measures of finger individuation correlated with the ARAT and the Moberg scores. This difference may relate to the fact that all our measures had a kinetic (force) component. Hence, finger individuation seems to be involved in grasp function, might usefully complement other functional scales, and its specific training may provide more efficient recovery than conventional rehabilitation [45].

### Limitations

The main limitation of our study concerns the group size: some findings (e.g. correlations between FFM measures and clinical scores) need to be confirmed with a larger sample that represents a broader range of lesion size and localization, as well as a more representative range of functional impairment. Nevertheless, even this restricted sample showed clear-cut group differences and provided individually diverse dexterity profiles. Two methodological limits of the FFM were identified in the present study: the sequential tapping task was too difficult and some patients had problems in maintaining their fingers on the FFM pistons, which may have affected certain performance measures. These issues will be addressed by simplifying the sequence task and by re-design of the FFM device.

### CONCLUSIONS

We developed a novel device, the FFM, to quantify key components of manual dexterity in a clinical setting. Use of the device, together with four visuo-motor tasks, was feasible in a group of hemiparetic stroke patients. On the group level, patients were significantly impaired in all four visuo-motor tasks compared to healthy control subjects. Patients showed less accurate finger force control, slowed finger tapping rate, more error in finger selection and in sequential finger tapping. Moreover, the four tasks allowed for individual profiling of post-stroke impairment in dexterity. This suggests that this new device provides a more complete and more sensitive assessment of manual dexterity than previous devices or clinical scores.

### REFERENCES

1. Heffner R S, Masterton R B: The role of the corticospinal tract in the evolution of human digital dexterity. Brain BehavEvol. 1983, 23 (3-4):165-83.
2. Lemon R N: Descending pathways in motor control. Annu Rev Neurosci. 2008, 31:195-218.
3. Napier, J R: Prehensility and opposability in the hands of primates. Symp. Zool. Soc. London 1961, 5:115-32.
4. Zatsiorsky V M, Latash M L: Multifinger prehension: an overview. J Mot Behav. 2008, 40(5):446-76.
5. Maier M A, Hepp-Reymond M C: EMG activation patterns during force production in precision grip. I. Contribution of 15 finger muscles to isometric force. Exp Brain Res. 1995, 103 (1): 108-22.
6. Renner C I, Bungert-Kahl P, Hummelsheim H: Change of strength and rate of rise of tension relate to functional arm recovery after stroke. Arch Phys Med Rehabil. 2009, 90(9):1548-56.
7. Ehrsson H H, Fagergren A, Jonsson T, Westling G, Johansson R S, Forssberg H: Cortical activity in precision-versus power-grip tasks: an fMRI study. J Neurophysiol. 2000, 83(1):528-36.
8. Nowak D A, Glasauer S, Hermsdörfer J: Force control in object manipulation—a model for the study of sensorimotor control strategies. NeurosciBiobehav Rev. 2013, 37(8):1578-86.
9. Häger-Ross C, Schieber M H: Quantifying the independence of human finger movements: comparisons of digits, hands, and movement frequencies. J Neurosci. 2000, 20(22):8542-50.
10. Lang C E, Schieber M H: Reduced muscle selectivity during individuated finger movements in humans after damage to the motor cortex or corticospinal tract. J Neurophysiol. 2004, 91 (4): 1722-33.
11. Repp B H, Su Y H: Sensorimotor synchronization: a review of recent research (2006-2012). Psychon Bull Rev. 2013, 20(3):403-52.
12. Andres F G, Gerloff C: Coherence of sequential movements and motor learning. J ClinNeurophysiol. 1999, 16(6):520-7.
13. Catalan M J, Honda M, Weeks R A, Cohen L G, Hallett M: The functional neuroanatomy of simple and complex sequential finger movements: a PET study. Brain. 1998, 121 (Pt 2):253-64.
14. Parker V M, Wade D T, Langton Hewer R: Loss of arm function after stroke: measurement, frequency, and recovery. IntRehabil Med. 1986, 8(2):69-73.
15. Kwakkel G, Kollen B J, van der Grond J, Prevo A J: Probability of regaining dexterity in the flaccid upper limb: impact of severity of paresis and time since onset in acute stroke. Stroke. 2003, 34(9):2181-6.
16. Colebatch J G, Gandevia S C: The distribution of muscular weakness in upper motor neuron lesions affecting the arm. Brain. 1989, 112 (Pt 3):749-63.
17. Boissy P, Bourbonnais D, Carlotti M M, Gravel D, Arsenault B A: Maximal grip force in chronic stroke subjects and its relationship to global upper extremity function. ClinRehabil. 1999, 13(4):354-62.
18. Lindberg P G, Roche N, Robertson J, Roby-Brami A, Bussel B, Maier M A: Affected and unaffected quantitative aspects of grip force control in hemiparetic patients after stroke. Brain Res. 2012, 1452:96-107.
19. Hermsdörfer J, Hagl E, Nowak D A, Marquardt C: Grip force control during object manipulation in cerebral stroke. ClinNeurophysiol. 2003, 114(5):915-29.
20. Hermsdörfer J1, Hagl E, Nowak D A: Deficits of anticipatory grip force control after damage to peripheral and central sensorimotor systems. Hum Mov Sci. 2004, 23(5):643-62.
21. Raghavan P, Krakauer J W, Gordon A M: Impaired anticipatory control of fingertip forces in patients with a pure motor or sensorimotor lacunar syndrome. Brain. 2006, 129 (Pt 6):1415-25.
22. Raghavan P, Petra E, Krakauer J W, Gordon A M: Patterns of impairment in digit independence after subcortical stroke. J Neurophysiol. 2006, 95(1):369-78.
23. Kim Y, Kim W S, Yoon B: The effect of stroke on motor selectivity for force control in single- and multi-finger force production tasks. NeuroRehabilitation. 2014, 34(3):429-35.
24. Calautti C, Jones P S, Persaud N, Guincestre J Y, Naccarato M, Warburton E A, Baron J C: Quantification of index tapping regularity after stroke with tri-axial accelerometry. Brain Res Bull. 2006, 70(1):1-7.
25. Calautti C, Jones P S, Guincestre J Y, Naccarato M, Sharma N, Day D J, Carpenter T A, Warburton E A, Baron J C. The neural substrates of impaired finger tapping regularity after stroke. Neuroimage. 2010, 50(1):1-6.
26. Shimoyama I, Ninchoji T, Uemura K: The finger-tapping test. A quantitative analysis. Arch Neurol. 1990, 47(6):681-4.
27. Boyd L A, Winstein C J. Impact of explicit information on implicit motor-sequence learning following middle cerebral artery stroke. PhysTher. 2003, 83 (11):976-89.
28. Hobart J C, Cano S J, Zajicek J P, Thompson A J: Rating scales as outcome measures for clinical trials in neurology: problems, solutions, and recommendations. Lancet Neurol. 2007, 6(12):1094-105.
29. Fleuren J F, Voerman G E, Erren-Wolters C V, Snoek G J, Rietman J S, Hermens H J, Nene A V: Stop using the Ashworth Scale for the assessment of spasticity. J NeurolNeurosurg Psychiatry. 2010, 81(1):46-52.
30. Chen H M, Chen C C, Hsueh I P, Huang S L, Hsieh C L: Test-retest reproducibility and smallest real difference of 5 hand function tests in patients with stroke. Neurorehabil Neural Repair. 2009, 23(5):435-40.
31. Pandyan A D, Vuadens P, van Wijck F M, Stark S, Johnson G R, Barnes M P: Are we underestimating the clinical efficacy of botulinum toxin (type A)? Quantifying changes in spasticity, strength and upper limb function after injections of Botox to the elbow flexors in a unilateral stroke population. ClinRehabil. 2002, 16(6):654-60.
32. Nowak D A, Hermsdörfer J: Grip force behavior during object manipulation in neurological disorders: toward an objective evaluation of manual performance deficits. MovDisord. 2005, 20(1): 11-25.
33. Van der Lee J H, De Groot V, Beckerman H, Wagenaar R C, Lankhorst G J, Bouter L M. The intra- and inter rater reliability of the action research arm test: a practical test of upper extremity function in patients with stroke. Arch Phys Med Rehabil. 2001, 82(1):14-9.
34. Chen H F, Lin K C, Wu C Y, Chen C L: Rasch validation and predictive validity of the action research arm test in patients receiving stroke rehabilitation. Arch Phys Med Rehabil. 2012, 93(6):1039-45.
35. Amirjani N, Ashworth N L, Gordon T, Edwards D C, Chan K M: Normative values and the effects of age, gender, and handedness on the Moberg Pick-Up Test. Muscle Nerve. 2007, 35(6):788-92.
36. Patel M R, Bassini L: A comparison of five tests for determining hand sensibility. J ReconstrMicrosurg. 1999, 15(7):523-6.
37. Logan F A: Errors in copy typewriting. J ExpPsychol Hum Percept Perform. 1999, 25(6):1760-1773.
38. Koh C L, Hsueh I P, Wang W C, Sheu C F, Yu T Y, Wang C H, Hsieh C L: Validation of the action research arm test using item response theory in patients after stroke. J Rehabil Med. 2006, 38(6):375-80.
39. Lang C E, Wagner J M, Dromerick A W, Edwards D F: Measurement of upper-extremity function early after stroke: properties of the action research arm test. Arch Phys Med Rehabil. 2006, 87(12): 1605-10.
40. Ye Y, Ma L, Yan T, Liu H, Wei X, Song R: Kinetic measurements of hand motor impairments after mild to moderate stroke using grip control tasks. J NeuroengRehabil. 2014, 11:84.
41. Tyrell C M, Helm E, Reisman D S: Learning the spatial features of a locomotor task is slowed after stroke. J Neurophysiol. 2014, 112(2):480-9
42. Lang C E, Schieber M H: Human finger independence: limitations due to passive mechanical coupling versus active neuromuscular control. J Neurophysiol. 2004, 92(5):2802-10.
43. Reilly K T, Hammond G R: Independence of force production by digits of the human hand. NeurosciLett. 2000, 290(1):53-6.
44. Lang C E, Schieber M H: Differential impairment of individuated finger movements in humans after damage to the motor cortex or the corticospinal tract. J Neurophysiol. 2003, 90(2):1160-70.
45. Thielbar K O, Lord T J, Fischer H C, Lazzaro E C, Barth K C, Stoykov M E, Triandafilou K M and Kamper D G: Training finger individuation with a mechatronic-virtual reality system leads to improved fine motor control post-stroke. Journal of NeuroEngineering and Rehabilitation 2014, 11:171
46. Benjamin P R, Staras K, Kemenes G: What roles do tonic inhibition and disinhibition play in the control of motor programs? Front Behav Neurosci. 2010, 4:30.

1. A method for evaluating manual dexterity in a subject, said method comprising assessing the performance of said subject in the three following tasks:
(i) The Finger Force-Tracking;
(ii) The Single finger tapping; and
(iii) The Multi-finger tapping,
wherein:
the Finger Force-Tracking comprises the steps of:
a) providing instructions to the subject to exert a defined force or “the target force” by a unique finger on a piston; and
b) measuring the force exerted on the piston.
the Single finger tapping comprises the steps of:
a) providing instructions to the subject to tap a piston with a specific finger at a defined rate; and
b) detecting the taps on the piston.
the Multi-finger tapping comprises the steps of:
a) providing instructions to the subject to tap one or more pistons with one or more fingers simultaneously; and
b) detecting the taps on the piston.

2. The method of claim 1, further comprising assessing the performance of said subject in a fourth task or “Sequential finger tapping”, said task comprising the steps of:
a) providing instructions to the subject to tap piston with the four fingers in a defined sequence;
b) detecting the taps on the piston; and
c) comparing the sequence of taps to the sequence of instructions.

3. The method of according to claim 1, wherein the Finger Force-Tracking task comprises instructing said subject to increase and/or decrease and/or maintain constant said force.

4. The method according to claim 1, wherein:
the Finger Force-Tracking task comprises a further step of calculating a tracking error as the root-mean-square error between the applied force and the target force; and/or
the Finger Force-Tracking comprises a further step of measuring the time between the instruction and the exertion of said force on the piston and/or a further step of measuring the time during which the force is exerted on said piston.

5. The method according to claim 1, wherein the Single finger tapping task comprises a further step of measuring the rate of tapping.

6. The method according to claim 1, wherein step b) of the Single finger tapping task comprises detecting taps by a finger other than the specific finger of step a) in the absence of a concomitant tap by said specific finger or the “unwanted taps” and/or detecting taps by a finger other than the specific finger of step a) concomitant with a tap by said specific finger or “overflow taps”.

7. The method of claim 6, wherein the Single finger tapping task comprises a further step of computing unwanted taps and/or computing overflow taps.

8. The method according to claim 1, wherein step a) of the Multi-finger tapping task comprises tapping with a single finger or a combination of two fingers, said combination being selected from the list consisting of index/middle finger, index/ring finger, index/little finger, middle finger/ring finger, middle finger/little finger, and ring finger/little finger.

9. The method according to claim 1, wherein the Multi-finger tapping task comprises a further step of repeating steps a) and b) n times, n being an integer between 0 and 64.

10. The method of claim 9, wherein the Multi-finger tapping task comprises a further step of comparing the tap sequences, wherein said comparison comprises computing correct and incorrect taps.

11. The method according to claim 9, wherein the Multi-finger tapping task comprises a further step of computing absent taps in response to instructions, and/or additional taps with other finger(s) than the one(s) of the task.

12. The method according to claim 2, wherein the Sequential finger tapping comprises the further steps of:
c) repeating steps a) and b) n times, wherein n is an integer comprised between 0 and 15; and
d) comparing the sequence of taps to the sequence of instructions.

13. The method according to claim 2, wherein the Sequential finger tapping comprises the further steps of:
c) repeating steps a) and b) n times, wherein n is an integer comprised between 0 and 15;
d) commanding the subject to tap said sequence of step a) without being provided any instruction before each tap of the said sequence;
e) detecting the taps on the pistons; and
f) comparing the sequence of taps to the sequence of instructions.

14. The method according to claim 12, wherein comparing the sequence of taps to the sequence of instructions comprises computing the correct sequences and the incorrect sequences, and optionally the number of consecutive correct taps within parts of an incorrect sequence.

15. The method according to claim 1, wherein each of the task is assessed by using the Finger Force Manipulandum (FFM).

16. A method for diagnosing an upper limb and/or hand impairment in a subject, said method comprising:
a) evaluating manual dexterity in said subject by the method of any one of the previous claims;
b) comparing the performance of said subject for each of the tasks with the performance of a healthy subject; and
c) diagnosing upper limb and/or hand impairment if performance in at least one of the tasks is below performance of said healthy subject.

17. A product/computer program containing a set of instructions characteristic of implementation of the method according to claim 1.

18. A processing system including a computation unit and an input interface, said system including means for implementing the method according to claim 1.
